(19) United States
(12) Patent Application Publication
(10) Pub. No.: US 2006/0161222 A1
(43) Pub. Date: Jul. 20, 2006

(54) Multiple band communications for an implantable medical device

(76) Inventors: Gregory J. Haubrich, Champlin, MN (US); Len D. Twetan, Excelsior, MN (US); George Rosar, Minneapolis, MN (US)

Correspondence Address: MEDTRONIC, INC., 710 MEDTRONIC PARK, MINNEAPOLIS, MN, 55432-9924, US

(21) Appl. No.: 11/035,518

(22) Filed: Jan. 15, 2005

(51) Int. Cl. A61N 1/08

(52) U.S. Cl. 607/60; 607/32

(57) ABSTRACT

In some embodiments, a medical device system may include one or more of the following features: (a) an external medical device (EMD), (b) an implantable medical device (IMD) disposed within a hermetically sealed housing adapted for implantation in the body of a patient to provide a therapy delivery and/or monitoring function, (c) two or more transmitters disposed within the EMD, the two or more transmitters uplinking telemetry transmissions generated by the EMD, (d) two or more receivers disposed within the IMD, the two or more receivers downlinking the telemetry transmissions, and (e) the IMD processing the telemetry transmission received by a selected one of the two or more receivers, the selection being based on which receiver is receiving the best quality signal transmission reception.

### FIELD

The present disclosure relates generally to telemetry systems for uplink and/or downlink telemetry transmission between an implantable medical device (IMD) and an external medical device (EMD) such as a programmer or monitor and more specifically to a method for selection of an optimal telemetry communications link.

### BACKGROUND

In the context of programming the operating modes or parameters of an IMD or in receiving information from an IMD, it is helpful to reduce interference and/or fades in the telemetry transmission between an IMD and the EMD. In most currently available systems, the programmer is placed in close proximity to the implanted device, typically by means of a telemetry head in contact with the patient's body. In such applications, there is little likelihood of interference and/or fades in the telemetry transmission.

More recently it has been proposed to provide communication systems for implantable devices in that the telemetry head communication occurs directly between the implanted medical device and a programmer or monitor which, may be located some distance from the patient. Such systems are disclosed in U.S. Pat. No. 5,404,877 issued to Nolan et al, and U.S. Pat. No. 5,113,869 issued to Nappholz. In the Nappholz patent, in particular, broadcasting RF signals from an implanted device to a programmer or monitor that may be located some feet away from the patient is suggested. Such a communication system is also disclosed in U.S. Pat. No. 6,240,317 for a “Telemetry System For Implantable Medical Devices”, filed Apr. 30, 1999 by Villaseca et al., which is incorporated herein by reference in its entirety. In use of such systems, the possibility of interference and/or fades in the telemetry transmission increases as they are frequency and spatially selective.

### SUMMARY

In some embodiments, a medical device system may include one or more of the following features: (a) an external medical device (EMD), (b) an implantable medical device (IMD) disposed within a hermetically sealed housing adapted for implantation in the body of a patient to provide a therapy delivery and/or monitoring function, (c) two or more transmitters disposed within one of the EMD and the IMD, the two or more transmitters uplinking telemetry transmissions generated by the one of the EMD and IMD, (d) two or more receivers disposed within the other of the one of the EMD and the IMD, the two or more receivers downlinking the telemetry transmissions, and (e) the other of the one of the EMD and the IMD processing the telemetry transmission received by a selected one of the two or more receivers, the selection being based on which receiver is receiving the best quality signal transmission reception.

In some embodiments, a medical device communication system may include one or more of the following features: (a) an external medical device (EMD) having a transmitter and a receiver for downlinking and uplinking telemetry transmissions respectively, and (b) a battery powered implantable medical device (IMD) disposed within a hermetically sealed housing adapted for implantation in the body of a patient to provide a therapy delivery and/or monitoring function, the IMD having a transmitter and a receiver for establishing full duplex communication with the EMD via downlinking and uplinking telemetry transmissions with the EMD over separate communication channels.

In some embodiments, a method of communication in a medical device system may include one or more of the following features: (a) uplinking two or more transmitters telemetry transmissions generated by the one of an EMD and an IMD being hermetically sealed and adapted for implantation in the body of a patient to provide a therapy delivery and/or monitoring function, (b) downliking the telemetry transmissions with two or more receivers within the other of the one of the EMD and the IMD, (c) processing the telemetry transmission received by a selected one of the two or more receivers with the other of the one of the EMD and the IMD, the selection being based on which receiver is receiving the best quality signal transmission reception, (d) checking periodically the signal quality received by the one or more receivers not selected by the other of the one of the EMD and the IMD, and (e) selecting a different receiver based on which receiver is receiving the best quality signal transmission if the signal quality received by the selected receiver deteriorates to beyond a threshold level.

### DRAWINGS

FIG. 1 is a simplified circuit block diagram of functional uplink and downlink telemetry transmission functions of an EMD and IMD in accordance with the present teachings.

FIG. 2 is a simplified circuit block diagram of functional blocks of the EMD of FIG. 1 in accordance with the present teachings.

FIG. 3 is a simplified circuit block diagram of functional blocks of the IMD of FIG. 1 in accordance with the present teachings.

FIG. 4 is a simplified schematic illustration of a telemetry system for programming an IMD in a clinical setting in accordance with an embodiment of a system according to the present invention.

FIG. 5 is a simplified circuit block diagram of functional blocks of a communication system in an embodiment of the present teachings.

FIG. 6 is a simplified circuit block diagram of functional blocks of a communication system in an embodiment of the present teachings.

FIG. 7 is a simplified circuit block diagram of functional blocks for a communication system in an embodiment of the present teachings.

FIG. 8 is a simplified circuit block diagram of functional blocks for a communication system in an embodiment of the present teachings.

FIG. 9 is a flowchart showing the operation of a medical device communication system in an embodiment of the present teachings.

### DESCRIPTION OF VARIOUS EMBODIMENTS

The following disclosure is made to enable a person skilled in the art to make and use the present teachings. Various modifications to the illustrated embodiments will be readily apparent to those skilled in the art, and the generic principles herein may be applied to other embodiments and applications without departing from the present teachings. Thus, the present teachings are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the present teachings. Skilled artisans will recognize the examples provided herein have many useful alternatives and fall within the scope of the present teachings.

The present teachings relate to a long-range telemetry system of the general type described in the above-referenced Villaseca et al. application wherein an implanted device may be programmed or monitored at a distance from the patient in whom the device is implanted. The system may employ RF transmission in an occupied band of about 402-405 MHz as in the Villaseca et al. application. Within this bandwidth, one or a number of communication channels may be available. Other frequency ranges may be substituted including frequency ranges up to several gigahertz. Each telemetry transmission may be formatted in a frame based format using frequency shift keying or other modulation format. The operating physical distance between the IMD antenna and the external device antenna is 0-2 meters and may be on the order of at 5-10 meters or more. The present teachings are discussed relating to a long range telemetry system, such as a system in compliance with Telemetry C, which permits communication over distances from 0-10 meters or even more as discussed in U.S. Pat. Nos. 6,456,887, 6,169,925, and 5,752,925 herein incorporated by reference in their entirety. However, the present teachings can be utilized with short range telemetry systems, such as Telemetry A or Telemetry B which typically require the telemetry head to be adjacent to or within a few meters of the implanted device as discussed in U.S. Pat. Nos. 6,223,083, 6,295,473, and 5,292,343 herein incorporated by reference in their entirety without departing from the spirit of the present teachings.

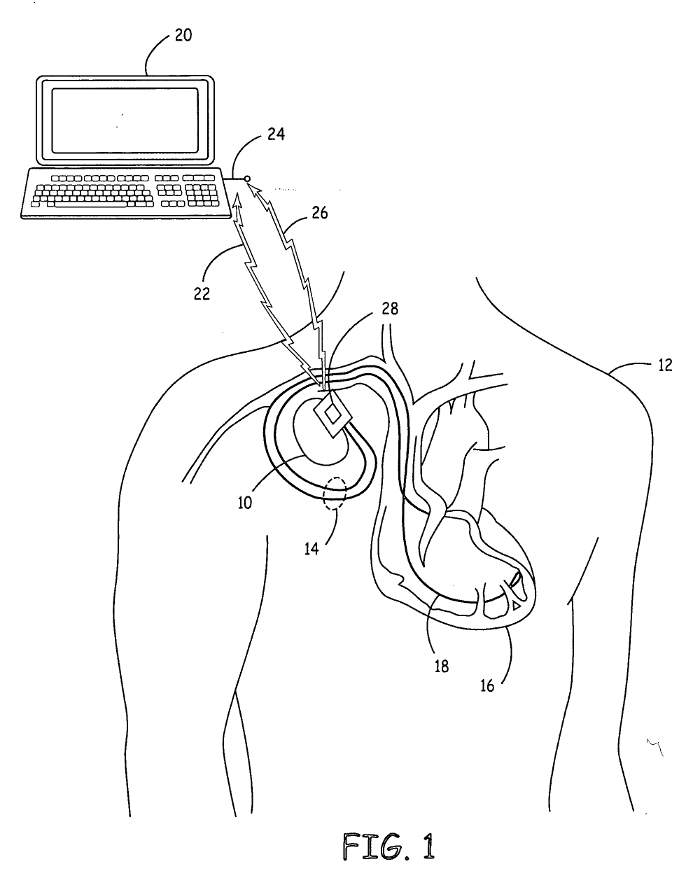

FIG. 1 is a simplified schematic diagram of functional uplink and downlink telemetry transmission functions allowing bi-directional telemetry communication between an END, e.g., an external programmer 20, and an IMD, e.g., a cardiac pacemaker IPG 10, in accordance with the present invention. The IPG 10 is implanted in the patient 12 with at least one cardiac pacing lead 18 in a manner known in the art. The IPG 10 contains an operating system that may employ a microcomputer or a digital state machine for timing sensing and pacing functions in accordance with a programmed operating mode. The IPG 10 also contains sense amplifiers for detecting cardiac signals, patient activity sensors or other physiologic sensors for sensing the need for cardiac output, and pulse generating output circuits for delivering pacing pulses to at least one heart chamber of the heart 16 under control of the operating system. The operating system includes memory registers or RAM for storing a variety of programmed-in operating mode and parameter values that are used by the operating system. The memory registers or RAM may also be used for storing data compiled from sensed cardiac activity and/or relating to device operating history or sensed physiologic parameters for telemetry out on receipt of a retrieval or interrogation instruction. All of these functions and operations are well known in the art, and many are employed in other programmable IMDs to store operating commands and data for controlling device operation and for later retrieval to diagnose device function or patient condition.

Programming commands or data are transmitted between an IPG RF telemetry antenna 28 and an external RF telemetry antenna 24 associated with the external programmer 20. In this configuration, it is not necessary that the external RF telemetry antenna 24 be contained in a programmer RF head of the type described above so that it can be located close to the patient's skin overlying the IPG 10. Instead, the external RF telemetry antenna 24 can be located on the case of the external programmer 20, and the programmer 20 can be located some distance away from the patient 12. For example, the external programmer 20 and external RF telemetry antenna 24 may be on a stand a few meters or so away from the patient 12. Moreover, the patient 12 may be active and could be exercising on a treadmill or the like during an uplink telemetry interrogation of real time ECG or physiologic parameters. The programmer 20 may also be designed to universally program existing IPGs that employ the conventional ferrite core, wire coil, RF telemetry antennas of the prior art and therefore also have a conventional programmer RF head and associated software for selective use with such IPGs. While the present disclosure relates to programmer 20 being some distance away from patient 12, it is fully contemplated the present teachings could be extended to prior programmers requiring a programming head to be in close proximity with the patient without departing from the spirit of the present teachings.

In an uplink telemetry transmission 22, the external RF telemetry antenna 24 operates as a telemetry receiver antenna, and the IPG RF telemetry antenna 28 operates as a telemetry transmitter antenna. Conversely, in a downlink telemetry transmission 26, the external RF telemetry antenna 24 operates as a telemetry transmitter antenna, and the IPG RF telemetry antenna 28 operates as a telemetry receiver antenna. Each RF telemetry antenna is coupled to a respective transmitter and/or receiver system.

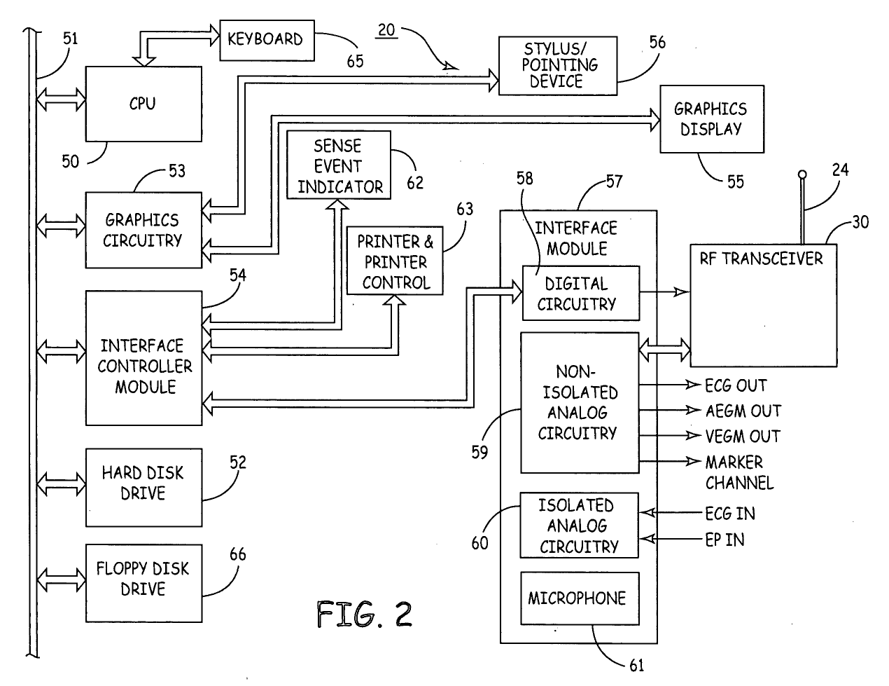

FIG. 2 is a simplified circuit block diagram of functional blocks of the external programmer 20 of FIG. 1. The external RF telemetry antenna 24 of the programmer 20 is coupled to a telemetry transmitter and/or receiver system block 30, which is discussed in more detail below. As shown in FIG. 2, programmer 20 is a personal computer type, microprocessor-based device incorporating a central processing unit 50, which may be, for example, an Intel 80386 or 80486 or Pentium microprocessor or the like. A system bus 51 interconnects CPU 50 with a hard disk drive 52 storing operational programs and data and with a graphics circuit 53 and an interface controller module 54. A floppy disk drive 66 or a CD ROM drive is also coupled to bus 51 and is accessible via a disk insertion slot within the housing of the programmer 20. Programmer 20 further comprises an interface module 57, which includes digital circuit 58, non-isolated analog circuit 59, and isolated analog circuit 60. Digital circuit 58 enables interface module 57 to communicate with interface controller module 54. Operation of the programmer in accordance with the present invention, is controlled by the microprocessor 50, as in turn controlled by software stored on disk drives 52 and/or 66 and/or by EPROM cartridges as described below.

In order for the physician or other caregiver or operator to communicate with the programmer 20, a keyboard 65 coupled to CPU 50 is optionally provided. However the primary communication mode may be through graphics display screen 55 of the well known “touch sensitive” type controlled by graphics circuit 53. A user of programmer 20 may interact therewith through the use of a stylus 56, also coupled to graphics circuit 53, which is used to point to various locations on screen 55 which display menu choices for selection by the user or an alphanumeric keyboard for entering text or numbers and other symbols. Various touch-screen assemblies are known and commercially available. The display 55 and or the keyboard 65 comprise means for entering command signals from the operator to initiate transmissions of downlink telemetry and to initiate and control telemetry sessions once a telemetry link with an implanted device has been accomplished. Graphics display screen 55 is also used to display patient related data and menu choices and data entry fields used in entering the data in accordance with the present invention as described below. Graphics display screen 55 also displays a variety of screens of telemetered out data or real time data.

Graphics display screen 55 may also display uplinked event signals as received and thereby serve as a means for enabling the operator of the programmer to correlate the receipt of uplink telemetry from an implanted device with the response-provoking event to the patient's body as disclosed above. Further handshaking functionality may be provided by a device such as microphone 61, which may be used to automatically detect tones generated by the IMD in a manner to be discussed below. Programmer 20 is also provided with a strip chart printer 63 or the like coupled to interface controller module 54 so that a hard copy of a patient's ECG, EGM, marker channel or of graphics displayed on the display screen 55 can be generated.

As will be appreciated by those of ordinary skill in the art, it is often desirable to provide a means for programmer 20 to adapt its mode of operation depending upon the type or generation of implanted medical device to be programmed. Accordingly, it may be desirable to have an expansion cartridge containing EPROMs or the like for storing software programs to control programmer 20 to operate in a particular manner corresponding to a given type or generation of implantable medical device. In addition, in accordance with the present invention, it is desirable to provide the capability through the expansion cartridge or through the floppy disk drive 66 or a CD ROM drive to expand or alter the formal generative grammars stored therein or in hard disk drive 52 as experience dictates the need or opportunity to do so.

The non-isolated analog circuit 59 and the digital circuitry 58 of interface module 57 is coupled to the transmitter/receiver system block 30 which is used to establish the uplink and downlink telemetry links between the IPG 10 and programmer 20. The atrial and ventricular sense amp circuits of IPG 10 may also be provided with (electrogram) EGM amplifiers that produce atrial and ventricular EGM signals. These A EGM and V EGM signals may be digitized and uplink telemetered to programmer 20 on receipt of a suitable interrogation command. The uplink telemetered EGM signals are received in telemetry transmission 22 and provided to non-isolated analog circuit 59. Non-isolated analog circuit 59, in turn, converts the digitized EGM signals to analog EGM signals (as with a digital-to-analog converter, for example) and presents these signals on output lines designated as A EGM OUT and V EGM OUT. These output lines may then be applied to a stripchart recorder 63 to provide a hard-copy printout of the A EGM or V EGM signals transmitted from IPG 10 for viewing by the physician. As these signals are ultimately derived from the intracardiac electrodes, they often provide different information that may not be available in conventional surface ECG signals derived from skin electrodes.

IPG 10 may also be capable of generating so-called marker codes indicative of different cardiac events that it detects. A pacemaker with marker channel capability is described, for example, in U.S. Pat. No. 4,374,382 to Markowitz, which patent is hereby incorporated by reference herein in its entirety. The markers provided by IPG 10 may be received by telemetry transmission 22 and presented on the MARKER CHANNEL output line from non-isolated analog circuit 59.

Isolated analog circuit 60 in interface module 57 is provided to receive external ECG and electrophysiological (EP) stimulation pulse signals. In particular, analog circuit 60 receives ECG signals from patient skin electrodes and processes these signals before providing them to the remainder of the programmer system in a manner well known in the art. Circuit 60 further operates to receive the EP stimulation pulses from an external EP stimulator for the purposes of non-invasive EP studies, as is also known in the art.

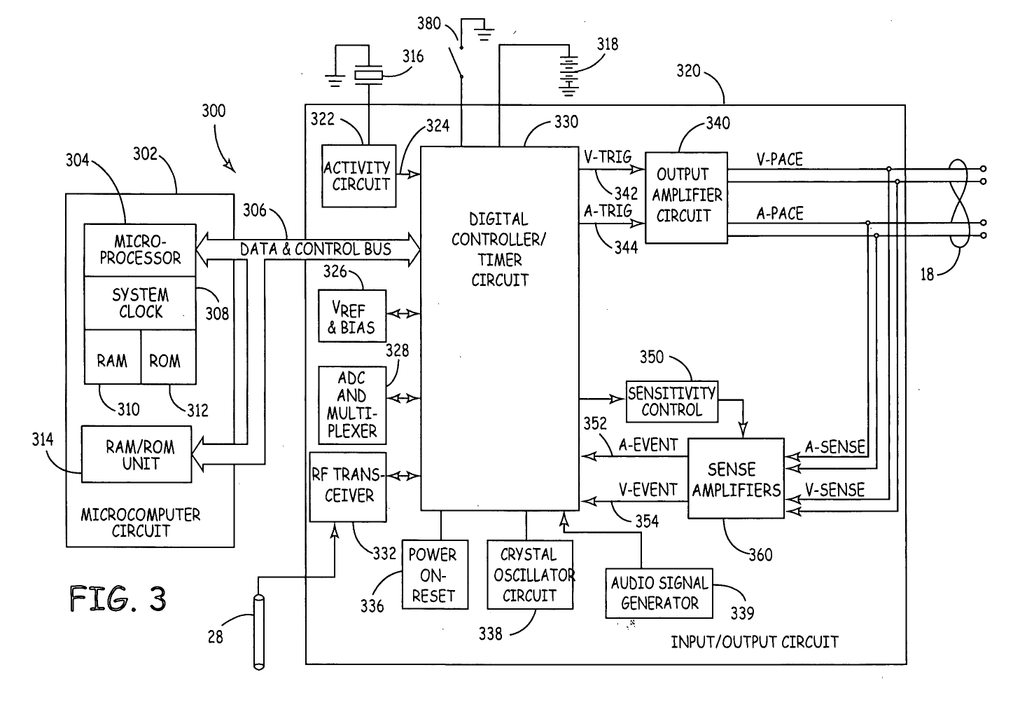

FIG. 3 is a simplified circuit block diagram 300 of functional blocks of IPG 10 of FIG. 1, which is an example of an IMD in which the present invention may be practiced. Uplink and downlink telemetry transmissions 22 and 26 are effected by the telemetry transceiver 332 that includes a telemetry transmitter and a telemetry receiver coupled with the IPG RF telemetry antenna 28. The telemetry transmitter and telemetry receiver are coupled to control circuitry and registers for compiling data and signals for uplink telemetry transmissions and for storing and decoding requests and commands embedded in downlink telemetry transmissions. The microcomputer 302 also stores and carries out the protocol governing the formatting of uplink telemetry transmissions and the timing and steps of carrying out the telemetry session protocols.

The IPG block diagram 300 is divided generally into a microcomputer circuit 302, an input/output circuit 320, and peripheral components including connectors for atrial and ventricular leads 18, the IPG RF telemetry antenna 28, a battery 318, an activity sensor 316 responsive to application of pressure and a magnetic field responsive solid state or reed switch 380. The block diagram 300 is fairly typical of prior art dual chamber pacemaker IPG circuits except for the specific configuration of the RF telemetry antenna, the transceiver 332 and the operating software for practicing the steps of the present invention.

The input/output circuit 320 includes a digital controller/timer circuit 330 coupled with a pulse generator output amplifier circuit 340, sense amplifiers 360, the IPG RF transceiver 332, other circuits and inputs described below and with a data and control bus 306 for communicating with the microcomputer circuit 302. The pulse generator circuit 340 includes a ventricular pulse generator circuit and an atrial pulse generator circuit, and the sense amplifier circuit 360 includes atrial and ventricular sense amplifiers adapted to be coupled to the atrium and ventricle of the patient's heart by means of leads 14. The output circuit 340 and sense amplifier circuit 360 may contain pulse generators and sense amplifiers corresponding to any of those presently employed in commercially marketed cardiac pacemakers.

Crystal oscillator circuit 338 provides the basic timing clock for the circuit, while battery 318 provides power. Power on reset circuit 336 responds to initial connection of the circuit to the battery for defining an initial operating condition and similarly, resets the operative state of the device in response to detection of a low battery condition. Reference and bias circuit 326 generates stable voltage reference and currents for the analog circuits within the input/output circuit 320. Analog to digital converter ADC and multiplexor circuit 328 digitizes analog signals and voltage to provide real time telemetry if a cardiac signals from sense amplifiers 360, for uplink transmission via RF transceiver circuit 332. Voltage reference and bias circuit 326, ADC and multiplexor 328, power on reset circuit 336 and crystal oscillator circuit 338 may correspond to any of those presently used in current marketed implantable cardiac pacemakers. Audio Signal Generator 339 may be provided to generate audible tones in response to telemetry downlink sessions initiated by the EMD.

Control of timing and other functions within the pacemaker circuit is provided by digital controller/timer circuit 330, which includes a set of timers and associated logic. Digital controller/timer circuit 330 defines the basic pacing interval of the IPG 10, which may take the form of an A-A escape interval initiated on atrial sensing or pacing and triggering atrial pacing at the expiration thereof or may take the form of a V-V escape interval, initiated on ventricular sensing or pacing and triggering ventricular pulse pacing at the expiration thereof. Digital controller/timer circuit 330 similarly defines the A-V escape intervals SAV and PAV. The microcomputer circuit 302 via data and control bus 306 controls the specific values of the intervals defined. Sensed atrial depolarization are communicated to the digital controller/timer circuit 330 on A event line 352, with ventricular depolarization communicated to the digital controller/timer circuit 330 on V event line 354. In order to trigger generation of a ventricular pacing pulse, digital controller/timer circuit 330 generates a trigger signal on V trigger line 342. Similarly, in order to trigger an atrial pacing pulse, digital controller/timer circuit 330 generates a trigger pulse on a trigger line 344.

Digital controller/timer circuit 330 also defines time intervals for controlling operation of the sense amplifiers in sense amplifier circuit 360. Typically, digital controller/timer circuit 330 will define an atrial blanking interval following delivery of an atrial pacing pulse, during which atrial sensing is disabled, as well as ventricular blanking intervals following atrial and ventricular pacing pulse delivery, during which ventricular sensing is disabled. Digital controller/timer circuit 330 will also define an atrial refractory period during which atrial sensing is disabled, this refractory period extending from the beginning of the A-V escape interval following either a sensed or paced atrial depolarization, and extending until a predetermined time following sensing of a ventricular depolarization or delivery of a ventricular pacing pulse. Digital controller/timer circuit 330 similarly defines a ventricular refractory period following ventricular sensing or delivery of a ventricular pacing pulse, which is typically shorter than the portion of the atrial refractory period following ventricular sensing or pacing. Digital controller/timer circuit 330 also controls sensitivity settings of the sense amplifiers 360 by means of sensitivity control 350.

Microcomputer circuit 302 controls the operational functions of digital controller/timer 324, specifying which timing intervals are employed, and controlling the duration of the various timing intervals, via data and control bus 306. Microcomputer circuitry contains a microprocessor 304 and associated system clock 308 and on processor RAM circuits 310 and 312, respectively. In addition, microcomputer circuit 302 includes a separate RAM/ROM chip 314. Microprocessor 304 is interrupt driven, operating in a reduced power consumption mode normally, and awakened in response to defined interrupt events, which may include delivery of atrial and ventricular pacing pulses as well as sensed atrial and ventricular depolarization. In addition, if the device operates as a rate responsive pacemaker, a timed interrupt, e.g., every two seconds, may be provided in order to allow the microprocessor to analyze the output of the activity circuit 322 and update the basic rate interval (A-A or V-V) of the device. In addition, the microprocessor 304 may also serve to define fixed or variable A-V escape intervals and atrial and ventricular refractory periods which may also decrease in duration along with decreases in duration of the basic rate interval. Similarly microprocessor 304 may define atrial and/or ventricular refractory periods which decrease in duration as a function of sensed or paced heart rate.

In FIG. 3, the IPG 10 is provided with the piezoelectric activity sensor 316, which is intended to monitor patient activity, in order to allow provision of rate responsive pacing, such that the defined pacing rate (A-A escape interval or V-V escape interval) increases with increased demand for oxygenated blood. Activity sensor 316 is typically mounted inside and against the IPG housing and is responsive to pressure waves or shocks transmitted to it through the patient's body. Activity sensor 316 normally generates electrical signals in response to sensed physical activity, namely shocks transmitted through the body from patient foot steps while walking or running, which are processed by activity circuit 322 and provided to digital controller/timer circuit 330. Activity circuit 332 and associated sensor 316 may correspond to the circuitry disclosed in U.S. Pat. No. 5,052,388, issued to Betzold et al., and U.S. Pat. No. 4,428,378, issued to Anderson et al. incorporated herein by reference in their entireties. In normal use, the activity circuit 322 operates in conjunction with software algorithms and programmed signal processing values in microcomputer 302 to derive an activity signal correlated to rate at which footsteps are sensed and to then adjust the pacing lower rate to the sensed patient activity level.

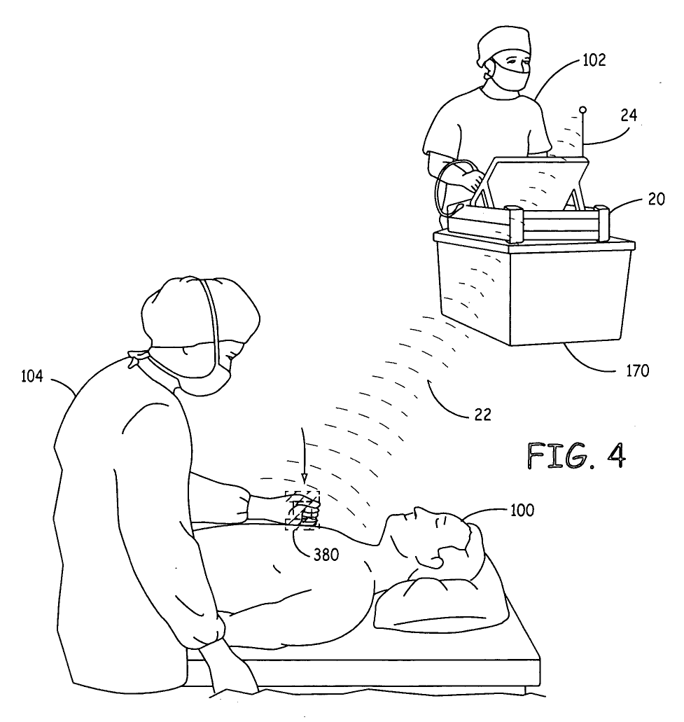

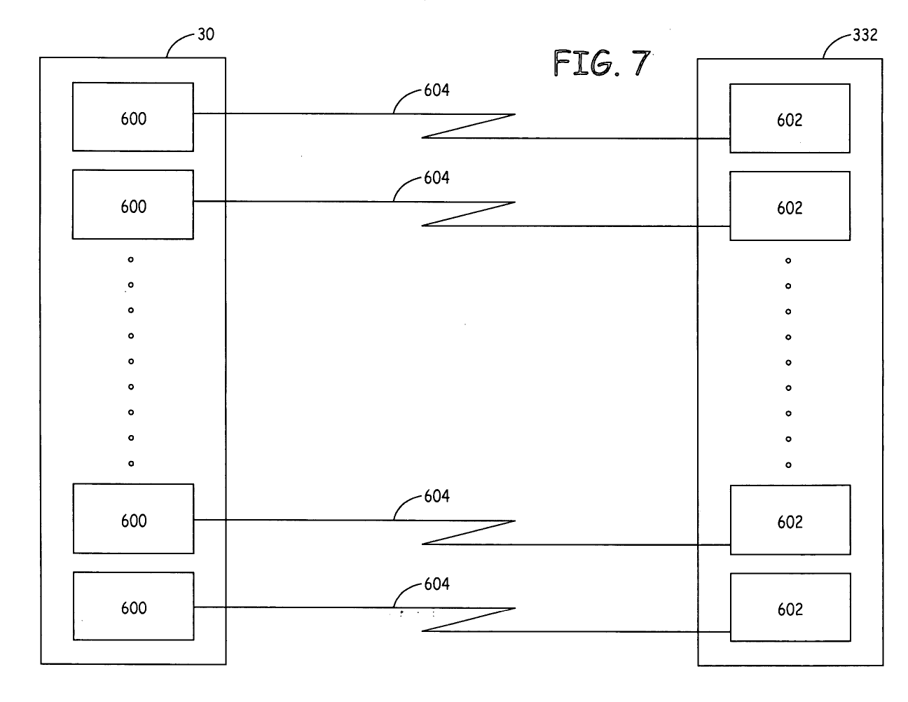

In one embodiment of the present invention illustrated in FIG. 4, the activity circuit 322 and activity sensor 316 of IPG 10 (or other IMD) may be used while the patient 100 is at rest to generate the implant event signal. After the IPG is placed in the ready state as described above, tapping the patient's skin over the implant site by the assistant 104 or the patient causes the activity sensor 316 to generate a sensor output signal which, in this context, is processed by activity circuit 322 and within digital controller/timer circuit 330 to develop the EMD discovery signal that is then encoded and transmitted via transmitter and/or receiver system 332 and IPG RF antenna 28 in an uplink telemetry transmission 22. Alternatively, a reed switch 380 could be held by assistant 104 or the patient that could cause the activity sensor 316 to generate a sensor output signal. The operator 102 observes the delivery of the tapping by the assistant 104 and the contemporaneous display of the implant event signal on the graphics display screen and/or sense event indicator 62 of the programmer 20 located at the somewhat remote station 170. It is simply necessary that the patient 100 remain seated or reclining during this initial verification phase prior to the commencement of the telemetry session. During the succeeding telemetry session, following verification, the patient 100 can be instructed to exercise to test the rate responsive operating mode and program differing rate control parameters and values. This technique, and these components, can be incorporated into other IMDs than rate responsive pacemakers and may be employed with other EMDs than the programmer 20, e.g., a bedside monitor for home use as illustrated in FIG. 7 described below or in clinical use, or in the context of re-programming an IMD in an office visit.

In FIG. 3, the IPG 10 is also provided with a solid state or reed switch 380 that is either opened or closed in response to an externally applied magnetic field. Conventionally, the magnetic field responsive switch 380 is employed to respond to the magnet in a conventional RF programming head for enabling the above-described closely coupled telemetry transmissions. The magnetic field responsive switch 380 in some embodiments of the present invention may be employed to initiate transmission of an event signal if activated by a magnetic field applied to the patient's body while the implanted device is in the ready state. In such embodiments the magnetic field responsive switch 380 may also be used to initially enable or “wake-up” the receiver in the IMD or to increase it's polling frequency.

The illustrated circuitry of FIGS. 2 and 3 is merely exemplary, and corresponds to the general functional organization of microcomputer controlled programmers and IMDs presently commercially available. It is believed that the present invention is most readily practiced in the context of such IMDs and EMDs, and that the present invention can therefore readily be practiced using software algorithms stored in RAM or ROM associated with the microcomputers. However, the present invention may also be usefully practiced by means of full custom integrated circuits, for example, a circuit taking the form of a state machine, in which a state counter serves to control an arithmetic logic unit to perform calculations according to a prescribed sequence of counter controlled steps. As such, the present invention should not be understood to be limited to a programmer and an IPG having an architecture as illustrated in FIGS. 2 and 3, and a circuit architecture as illustrated in FIGS. 2 and 3 is not believed to be a prerequisite to enjoying the benefits of the present invention.

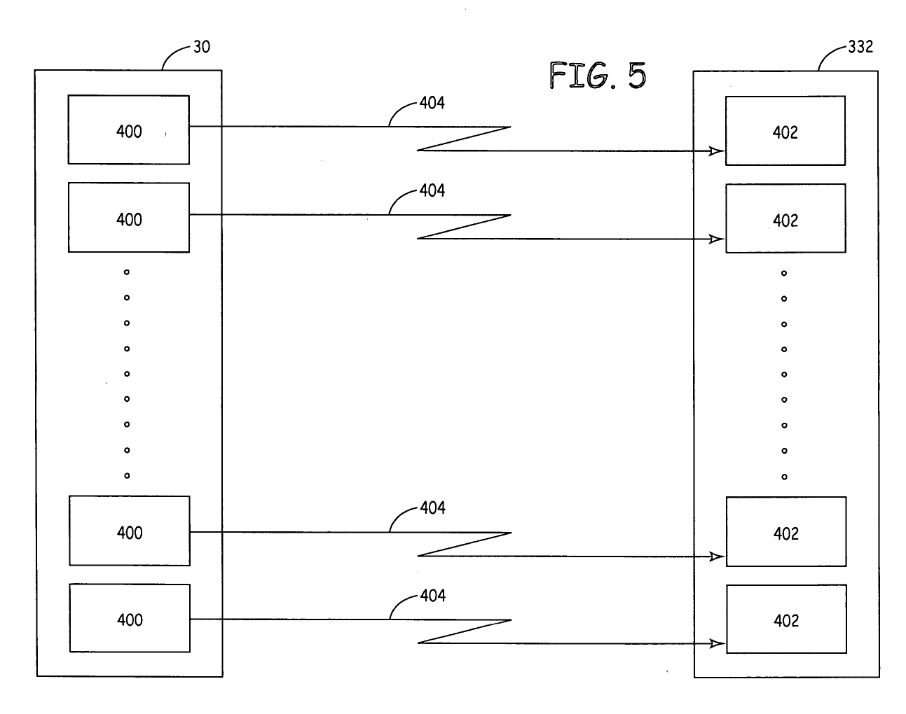

With reference to FIG. 5, a simplified circuit block diagram of functional blocks of a communication system in an embodiment of the present teachings is shown. Transmitter and/or receiver systems 30 and 332 are labeled transceivers for the purpose of the description as they can house transmitters, receivers, and/or transceivers without departing from the spirit of the present teachings. Transmitter and/or receiver system 30 houses two or more transmitters 400 for transmitting the uplink telemetry data. Transmitter and/or receiver system 332 houses two or more receivers 402 for receiving the downlink telemetry transmission. Telemetry transmitters 400 and telemetry receivers 402 are coupled to control circuitry and registers operated under the control of a microcomputer and software as described in the incorporated, commonly assigned patents. Telemetry transmitter 400 and telemetry receiver 402 can be coupled to control circuitry and registers operated under the control of a microcomputer and software as described in the incorporated, commonly assigned patents and pending applications.

Transmitters 400 can transmit the telemetry data through antenna 24, however, EMD 20 may be equipped with a compatible antenna or set of antennas that are arranged to avoid nulls or dead spots in reception, for example corresponding generally to that disclosed in the above-cited Villaseca et al. application or in U.S. Pat. No. 6,167,312 titled a “Telemetry System For Implantable Medical Devices” by Geodeke et al., which application is also incorporated herein by reference in its entirety. Transmission can be accomplished through time or phase multiplexing or any other multiplexing communications technology without departing from the spirit of the invention. Further, multiple antennas can be used, one for each transmitter, to avoid having to multiplex the transmissions. Receivers 402 can then receive transmissions 404 through antenna 28. IPG 10 may also employ, for example, an elongated antenna which projects outward from the housing of the IMD, as described in the cited Villaseca et al. application or may employ a coil antenna located external to the device housing as described in U.S. Pat. No. 6,009,350 issued to Renken, incorporated herein by reference in its entirety. Similar to EMD 20, IPG 10 could also use multiple antennas to avoid de-multiplexing of transmissions 404.

Transmitters 400 transmit uplink telemetry data to IPG 10 through transmissions 404. Transmitters 400 can be most any type of transmitter without departing from the spirit of the present teachings. Each transmitter 400 transmits generally the same data at generally the same time. However, each transmitter 400 transmits at a different frequency, which is discussed in more detail below. Receivers 402 receive transmissions 404. In some embodiments, each receiver 402 is paired to a transmitter 400, that is to say that each receiver 402 is adapted to receive the frequency of a transmitter 400. Receivers 402 can be most any type of receiver without departing from the spirit of the present teachings. Receivers 400 can then route the transmission data to microcomputer 302 through control bus 306. Microcomputer 302 then begins to process the transmission data as discussed above. Further, microcomputer 302 also begins to evaluate each incoming telemetry transmission for the transmission quality.

Microcomputer 302 utilizes software algorithms to evaluate the quality of the incoming transmissions 404 so IPG 10 can select an optimal (or best performing) telemetry communication link. In some embodiments, each transmission 404 has its own distinct frequency separated from other transmissions by a channel outside the band to insure no interference with the other transmissions 404. It is also helpful if transmissions 404 cover a wide range of frequencies to insure that any environmental effects or disturbances occurring in one frequency band does not affect any other frequency band due to frequency separation. The transmission quality assurance can be performed in a variety of ways including but not limited to determining the transmission with the largest signal strength, determining the transmission with the lowest data error signal to noise ratio, or utilizing other well known communication statistics. Once microcomputer 302 has made a determination which transmission has the best quality, then microcomputer 302 selects that receiver 402 as the primary input for all downlink telemetry transmissions. Establishing an initial telemetry communication session can happen in many forms including but not limited to simultaneous transmission or paired transmission and reception. Microcomputer 302 continues to monitor all of transmissions 404, continuously evaluating the quality of transmissions 404. If the selected transmission deteriorates in quality, microcomputer can instantly switch to the transmission having the best quality. This helps to insure the highest quality transmission is being received and helps to reduce frequency interference and fades. Additionally, microcomputer 302 could combine multiple receivers 402 and selects the highest frequency of data bits. This could be accomplished utilizing an algorithm such as

$$( \frac{m + 1}{2 m + 1} )$$

to select a predetermined number of receivers 402 and obtain the highest frequency of data bits with an algorithm. This algorithm is generally a voting scheme for microcomputer to choose 2 out of 3, 3 out of 5, or 4 out of 7 receivers.

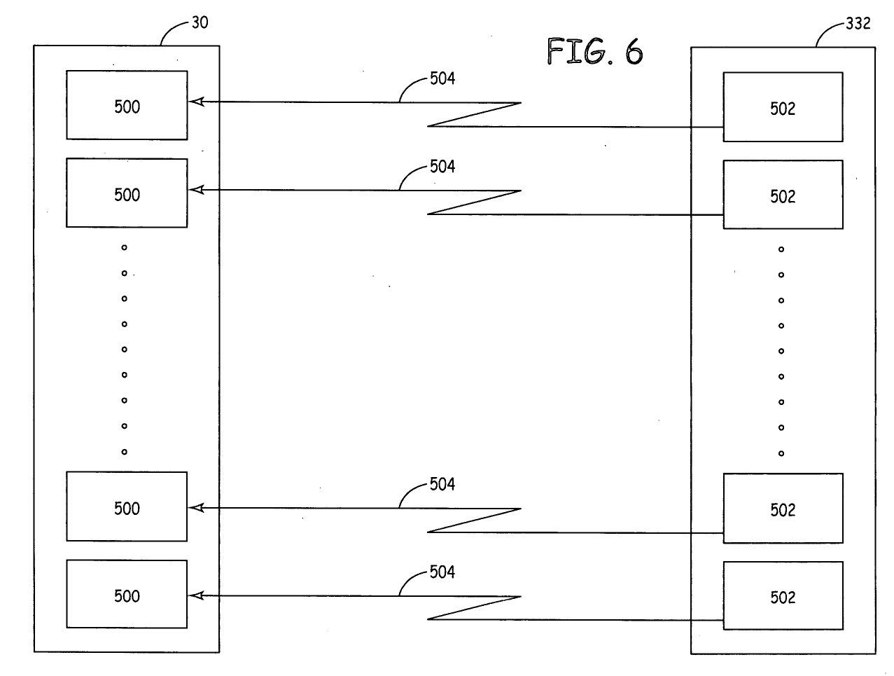

With reference to FIG. 6, a simplified circuit block diagram of functional blocks of a communication system in an embodiment of the present teachings is shown. In some embodiments, transmitter and/or receiver system 30 of EMD 20 houses receivers 500, while transmitter and/or receiver system 332 of IPG 10 houses transmitters 502. Transmitters 502 transmit uplink telemetry data to EMD 20 through transmissions 504. Similar to FIG. 5, each transmitter 502 transmits generally the same data at generally the same time. However, each transmitter 502 transmits at a different frequency. Receivers 500 receive transmissions 504. Similar to above, each receiver 500 is paired to a transmitter 502. Receivers 500 can then route the transmission data to CPU 50. CPU 50 can then begin to process the transmission data. And, similar to above, CPU 50 can also begin to evaluate each incoming telemetry transmission 504 for the transmission quality.

Similar to microcomputer 302, CPU 50 utilizes software algorithms to evaluate the quality of the incoming transmissions 504 so EMD 10 can select an optimal (or best performing) telemetry communication link. Similar to above, each transmission 504 has its own distinct frequency separated from other transmissions by a channel outside the band to insure no interference with the other transmissions 504. The transmission quality assurance can be performed similar to above. Once CPU 50 has made a determination which transmission has the best quality, then CPU 50 can select that receiver 500 as the primary input for all downlink telemetry transmissions 504. CPU 50 continues to monitor all of transmissions 504 continuously evaluating the quality of transmissions 504. If the selected transmission deteriorates in quality, CPU 50 can instantly switch to the transmission having the best quality.

With reference to FIG. 7, a simplified circuit block diagram of functional blocks for a communication system in an embodiment of the present teachings is shown. In some embodiments, transmitter and/or receiver system 30 of EMD 20 and transmitter and/or receiver system 332 of IPG 10 both house transceivers 600 and 602 respectively. Transceivers 600 and 602 both contain a transmitter for transmitting uplink telemetry transmissions and a receiver for receiving downlink telemetry transmissions. In some embodiments, either EMD 20 or IPG 10 can make the determination which transmission has the best quality and thus will be utilized. This decision could me made before implantation of IPG 10 or during programming by EMD 20 and the clinician could make this determination. It is further contemplated both EMD 20 and IPG 10 could communicate and make a determination together which transmission 604 has the best quality between both transceiver 600 and transceiver 602. For example, if communication 604 between a particular transceiver pair was rated the best quality by CPU 50, yet, microcomputer 302 rated the same communication 604 as being the second or third best quality 604, then microcomputer 302 and CPU 50 could agree to use that pair of transceivers since the overall quality of the communication is fairly high. Additionally, once a transmission frequency was selected, the transmitters in transceivers 602 (or alternatively transceivers 600) could be turned off to consume power. The receivers in transceivers 602 would continue to receive transmissions 604 from transceivers 600 and microcomputer 302 could continue to evaluate the incoming transmissions. If the quality of the selected receiver in transceiver 602 should deteriorate beyond the quality of the next best transmission quality, then microcomputer 302 could switch the reception of transmission 604 to the new receiver and begin transmitting from the newly selected transceiver 602. CPU 50 would identify it is receiving transmission 604 at a new frequency and switch it's selected transceiver 600 accordingly.

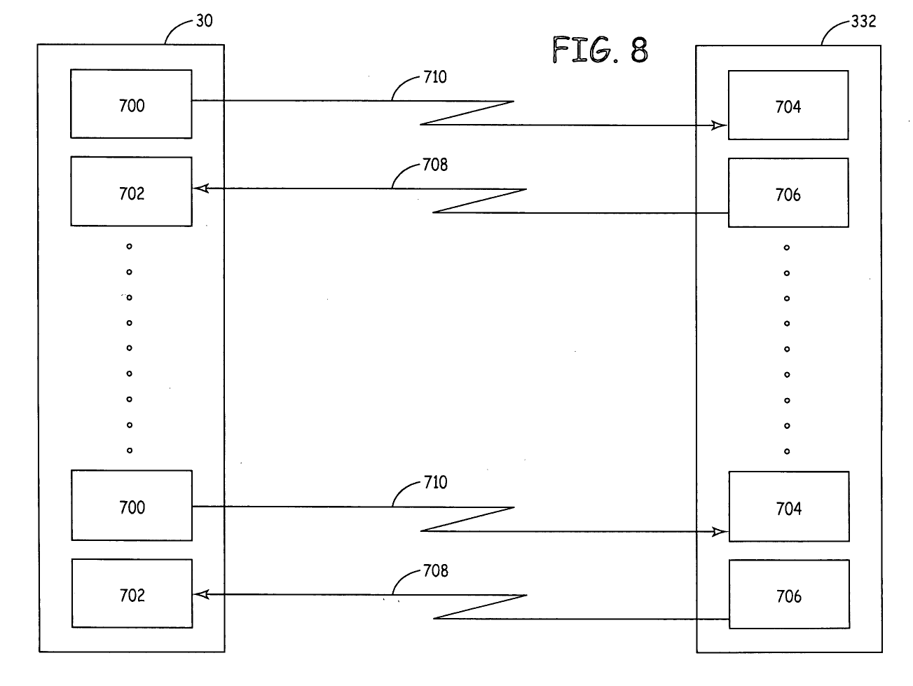

With reference to FIG. 8, a simplified circuit block diagram of functional blocks for a communication system in an embodiment of the present teachings is shown. In some embodiments, transmitter and/or receiver system 30 of EMD 20 houses two or more transmitters 700 and receivers 702 and transmitter and/or receiver system 332 of IPG 10 houses two or more transmitters 706 and receivers 704. In this full duplex structure each transmitter 700 & 706 transmits at a different frequency to its respective receiver 702 & 704. CPU 50 can then determine which transmission 708 has the highest quality coming from transmitters 706 similar to that discussed above. Microcomputer 302 can also determine which transmission 710 from transmitters 700 has the highest quality. In an embodiment, EMD 20 and IPG 10 relay the frequency of highest quality to one another. Thus when IPG 10 learns what frequency EMD 20 has chosen, IPG 10 can quit transmitting all the other frequencies to conserve battery power. EMD 20 could continue to transmit at all frequencies so IPG could continuously monitor the frequency with the highest quality and switch frequencies if necessary. It is contemplated that if IPG 10 needs to switch frequencies, IPG 10 begins transmitting from all its transmitters 706 to give EMD 20 an opportunity to switch frequencies if needed.

In an embodiment, each of EMD 20 and IPG 10 could have one transmitter 700 & 706 and one receiver 702 & 704. During initial transmissions each transmitter 700 & 706 would transmit over a plurality of channels each having different frequencies. CPU 50 and microcomputer 302 could then evaluate transmissions 708 and 710 for quality and select a frequency based upon quality. The respective frequency chosen by CPU 50 and microcomputer 302 would then be sent back to IPG 10 and EMD 20. If either EMD 20 or IPG 10 were to have trouble with their selected frequencies then the selection process would be repeated and a new frequency(s) would be selected. In the embodiment shown in FIG. 8, the full duplex allows IPG 10 and/or EMD 20 to halt a transmission quickly if the signal deteriorates. IPG 10 and/or EMD 20 would not have to wait until the transmission completed, as in half-duplex, and request retransmission of the data. A full duplex embodiment allows IPG 10 and/or EMD 20 to halt a transmission and retransmit on the same channel or a different channel.

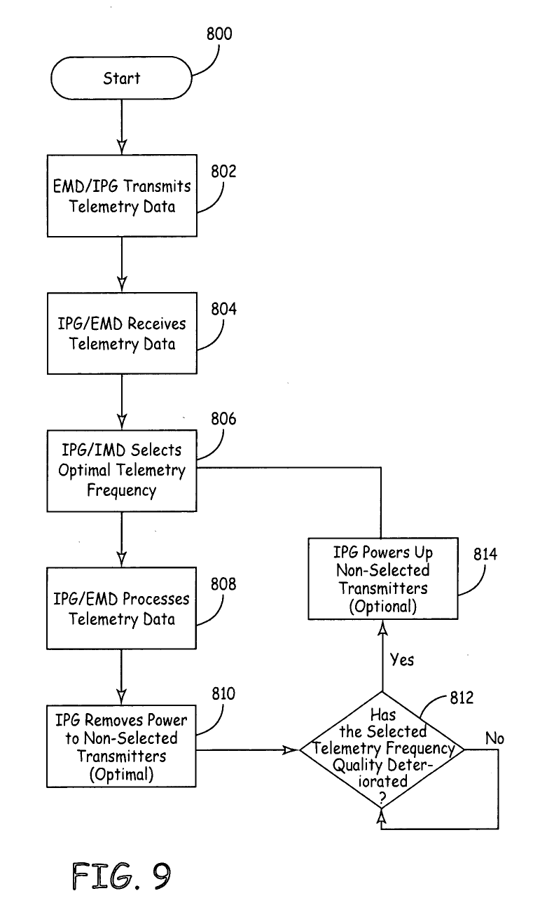

With reference to FIG. 9, a flowchart showing the operation of a medical device communication system in an embodiment of the present teachings is shown. In some embodiments, the medical device communication system begins at state 800. When necessary or needed EMD 20 or IPG 10 initiates transmitting telemetry session using data generated by EMD 20 or IPG 10 over at least two transmitters having separate frequencies at state 802. IPG 10 or EMD 20 can then receive the multiple frequencies with at least two receivers at state 804. IPG 10 and/or EMD 20 can select the frequency with an optimal quality at state 806. As discussed above, the determination of frequency quality can be based on several characteristics. After IPG 10 and/or EMD 20 have made a determination on which frequency has the best quality, IPG 10 and/or EMD 20 can then process the received telemetry data. Optionally, IPG can then remove power to all the transmitters transmitting the non-selected frequency at step 810. This is helpful in conserving IPG battery power. EMD 20 and/or IPG 10 can then continue to periodically sample and test all the transmitted frequencies for quality. If the quality of the selected frequency has not deteriorated, then IPG 10 or EMD 20 continue to periodically sample and test all the transmitted frequencies for quality. If the quality of the selected transmission has deteriorated (state 812), then IPG 10 and/or EMD 20 can determine the frequency with the best quality at state 806 and then process the data at state 808. Optionally, at state 814, IPG 10 can re-initiate power to its transmitters. This allows EMD to again evaluate the quality of all the transmitted frequencies from IPG 10 and select a new frequency if necessary.

Thus, embodiments of the MULTIPLE BAND COMMUNICATIONS FOR AN IMPLANTABLE MEDICAL DEVICE are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A medical device system, comprising:
an external medical device (EMD);
an implantable medical device (IMD) two or more transmitters disposed within one of the EMD and the IMD, the two or more transmitters uplinking telemetry transmissions generated by the one of the EMD and IMD;
two or more receivers disposed within the other of the one of the EMD and the IMD, the two or more receivers downlinking the telemetry transmissions, and
the other of the one of the EMD and the IMD processing the telemetry transmission received by a selected one of the two or more receivers, the selection being based on which receiver is receiving the best quality signal transmission reception.

2. The medical device system of claim 1, wherein the EMD is an external programming unit.

3. The medical device system of claim 1, wherein the EMD is a patient monitor.

4. The medical device system of claim 1, wherein the telemetry transmissions include therapy delivery and monitoring operational mode programming data for the IMD.

5. The medical device system of claim 1, wherein the telemetry transmissions include patient data developed from the IMD transmitted in real-time.

6. The medical device system of claim 1, wherein one or more of the transmitters uplink telemetry transmissions via a modulated RF transmission.

7. The medical device system of claim 1, wherein the two or more transmitters uplink telemetry transmissions over different frequency bands.

8. The medical device system of claim 1, wherein the two or more receivers are tuned to receive telemetry transmissions from a respective transmitter.

9. The medical device system of claim 1, wherein the two or more transmitters uplink telemetry transmissions via a single antenna.

10. The medical device system of claim 1, wherein the two or more transmitters concurrently uplink the same telemetry transmissions.

11. A medical device communication system, comprising:
an external medical device (EMD) having a transmitter and a receiver for downlinking and uplinking telemetry transmissions respectively; and
a battery powered implantable medical device (IMD) having a transmitter and a receiver for establishing full duplex communication with the EMD via downlinking and uplinking telemetry transmissions with the EMD over separate communication channels.

12. The medical device system of claim 11, wherein the telemetry transmissions include therapy delivery and monitoring operational mode programming data for the IMD.

13. The medical device system of claim 11, wherein the transmitters uplink telemetry transmissions in a frequency range between about 175 kHz and several gigahertz.

14. The medical device system of claim 11, wherein the EMD selects the communication channel and then initiates a telemetry session.

15. A method of communicating in a medical device system, comprising:
uplinking two or more transmitters telemetry transmissions generated by the one of an external medical device (EMD) and an implantable medical device (IMD), for downlinking the telemetry transmissions with two or more receivers within the other of the one of the EMD and the IMD; and
processing the telemetry transmission received by a selected one of the two or more receivers with the other of the one of the EMD and the IMD, the selection being based on which receiver is receiving the best quality signal transmission reception.

16. The method of claim 15, wherein the telemetry transmissions include patient data transmitted in real-time.

17. The method of claim 15, wherein the two or more transmitters uplink telemetry transmissions over different frequency bands.

18. The method of claim 15, wherein one or more of the transmitters uplink telemetry transmissions in a frequency range between about 175 kHz and several gigahertz.

19. The method of claim 15, further comprising the step of checking periodically the signal quality received by the one or more receivers not selected by the other of the one of the EMD and the IMD.

20. The method of claim 19, further comprising the step of selecting a different receiver based on which receiver is receiving the best quality signal transmission if the signal quality received by the selected receiver deteriorates to beyond a threshold level.
